(19) United States
(12) Patent Application Publication
(10) Pub. No.: US 2020/0309766 A1
(43) Pub. Date: Oct. 1, 2020

(54) CHEMOPREDICTIVE ASSAY FOR RECURRENT CHEMOTHERAPY

(71) Applicant: OROC, Bay Village, OH (US)

(72) Inventor: Saubhik SENGUPTA, Kolkata (IN)

(21) Appl. No.: 16/835,220

(22) Filed: Mar. 30, 2020

Related U.S. Application Data
(60) Provisional application No. 62/826,746, filed on Mar. 29, 2019.
(60) Provisional application No. 62/826,752, filed on Mar. 29, 2019.

(51) Int. Cl. G01N 33/50; C12N 5/09

(52) U.S. Cl. CPC G01N 33/5011; C12N 5/0693

(57) ABSTRACT

A chemopredictive assay, including culturing cancer cells of interest; exposing the cancer cultures to several chemotherapeutic agents; identifying the most effective chemotherapeutic agent; culturing surviving cancer cells to prepare second cultures; exposing the second cultures to several chemotherapeutic agents; and identifying the most effective chemotherapeutic agent for treating recurrent cancer.

### BACKGROUND

The present invention relates to predictive assays for chemotherapy, more specifically the present invention relates to predictive assays for screening chemotherapeutic agents for efficacy in the treatment of naive, treated metastatic and recurrent solid tumor cancers (breast, lung, head and neck, thyroid, parathyroid, colon and colorectal, esophageal, gastric, gall bladder, pancreas, lymphomas, ovarian and primary peritoneal, vulvar, vaginal, and cervical, urinary bladder, liver).

Chemotherapy relates to the treatment of cancer with drugs that preferentially kill cancer cells. Typically, the chemotherapeutic agent selective by virtue of having a higher toxicity in cells that divide rapidly, such as cancer cells.

The selection of the correct chemotherapeutic agent for treatment is often of great importance, and may take into consideration factors such as the toxicity of the agent, the type of cancer under treatment, and the type and severity of potential side effects of the selected agent and the data of the available clinical trials.

In addition, a chemotherapeutic agent may be selected for an individual patient based upon the specific genetic and phenotypical characteristics of the patients' tumor. This tailored approach may result in a chemotherapy regimen that is both less toxic and more effective for a given individual. Clinical assays that are used to select a chemotherapeutic agent in this way are referred to as chemopredictive assays.

Chemopredictive assays are typically used to select a first-line chemotherapeutic agent. In some cases cancer will recur after an initial therapy. In such instances a different chemotherapeutic agent is typically selected for an additional treatment regimen, in the belief that the recurring tumors will have developed at least some degree of resistance to the first-line chemotherapeutic agent used previously. Unfortunately, there are currently no clinical tools that can be used to accurately predict the best second-line drug for a particular patient. NCCN guidelines of 2018 strictly prohibit the use of these testing strategies for recurrent cases due to lack of data of efficacy of these tests in second line management.

The present disclosure is directed to a chemopredictive assay useful for the selection of chemotherapeutic agents to treat naïve, treated, metastatic and recurrent solid tumor cancers.

### SUMMARY

The present invention is directed to chemopredictive assays, where the assay includes culturing cancer tissues of interest; exposing the cancer tissue cultures to several chemotherapeutic agents treated in liver organoids (chemotherapy agents treated in liver organoids potentially generate active molecules in the body, as opposed to the drugs given directly to cancer tissues); identifying the most effective chemotherapeutic agent; culturing surviving cancer cells to prepare second cultures; exposing the second cultures to different tissue organoids created in the laboratory to create a metastatic scenario, followed by challenge with several chemotherapeutic agents treated in liver organoids; and identifying the most effective chemotherapeutic agent for treating recurrent cancer.

### BRIEF DESCRIPTION OF THE DRAWINGS

FIG. 1 is a flowchart for a method of screening chemotherapeutics for second-line chemotherapy, according to a representative embodiment of the invention.

### DETAILED DESCRIPTION

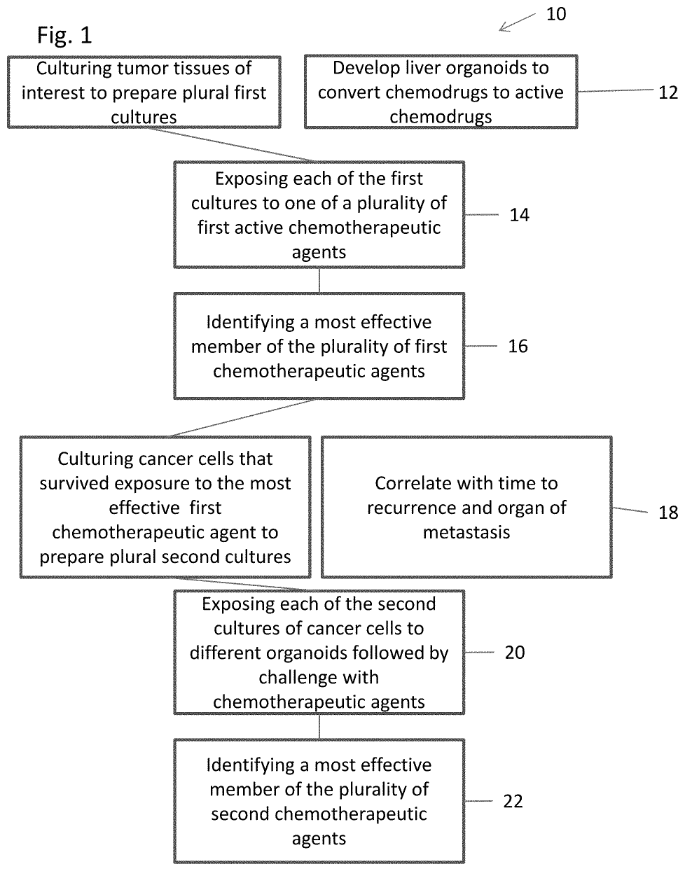

The present chemopredictive assay includes an a) screening process for chemotherapeutic agents, where the screening process determines the effectiveness of the chemotherapeutic agents against naïve, treated metastatic or recurring cancer cells and b) identify target organs of metastasis and time to recurrence. As set out in FIG. 1, a representative example of a method of the present invention is depicted in flowchart 10, and includes culturing liver organoids and exposing each of the chemo-drugs to the liver organoids to generate active drugs at 12, followed by using these drugs to treat tumors of patients at 14, identifying a most effective member of the plurality of first chemotherapeutic agents at 16, culturing cancer cells that survived exposure to the most effective first chemotherapeutic agent to prepare plural second cultures at 18, exposing each of the second cultures of cancer cells to different organoids followed by second chemotherapeutic agents at 20, and identifying a most effective member of the plurality of second chemotherapeutic agents at 22. At 18, time taken by the organoids to home cancer cells will be directly correlated with patients time to recurrence to generate a predictive model for identifying time to recurrence for end stage management, and the organ where cancer cells home fastest will be directly correlated with the patients' metastatic organ, predicting organ of metastasis.

In general, the present assay is performed under conditions selected to mimic the environment in which the cancer cells of interest exist, optionally including an extracellular matrix and/or a monolayer of normal cells upon a selected substrate. In this environment, selected tumor cells are challenged with multiple chemotherapeutic candidate drugs and a first-line selection of chemotherapy agent is performed, for example by direct histopathology.

Subsequent second-line chemotherapeutic selection is performed by assessing the ability of cells that were exposed to the first-line chemotherapeutic agent to grow into secondary colonies, and their ability to grow in the organoid followed by exposure to a second-line chemotherapeutic agent. The second-line selection of chemotherapeutic agent is based upon the ability of the surviving cancer cells to remain viable after exposure to a variety of second-line chemotherapeutic agents.

Substrate. Cell colonies, either of normal cells or of cancer cells, are typically prepared upon some type of supporting substrate. The substrate may be as basic as the surface of a microwell plate. However, the predictive value of the present screening method may be enhanced by preparing a substrate that more closely resembles the environment within the patient.

In one aspect, the substrate includes a matrix, typically an organic matrix. The matrix may be composed of one or more biological polymers. The matrix may include proteins, and may be a solid or semi-solid matrix. In one embodiment, the matrix includes MATRIGEL, a gelatinous mixture of proteins (BD Biosciences) or hydrogel.

The substrate may be further enhanced by preparing an environment of normal cells collected from the vicinity of the collected cancer cells. For example, normal cells may be cultured in order to prepare a substrate that includes at least a monolayer of normal cells.

The chemotherapeutic agents under evaluation in the present screening process may include any agent of interest selected by the physician. Typically the chemotherapeutic agent will be a drug that has been recognized as having efficacy in chemotherapy. In one embodiment of the invention, the chemotherapeutic agents being screened includes one or more of paclitaxel, carboplatin, cisplatin, adriamycin, gemcitabine, topotecan, etoposide, docataxel, ifosamide, and 5-fluoro uracil.

### EXAMPLES

### Example 1. Procurement of Tissue: Sample

A. Tumor/malignant cell sample:
I. To be collected at the time of core needle biopsy or surgical biopsy, or paracentesis/ascites samples in 10 ml RPMI 1640 medium (without FBS, Penicillin-Streptomycin).
II. Ascites sample: Ascites collection bottle containing peritoneal washing in 0.9% NaCl solution (Normal Saline) will be taken. This will be transported to the laboratory.
III. Solid tumor sample will be taken with a new sterile blade.

B. 10 ml whole blood in clotted vial will be collected from the ante-cubital vein.

Collection Media:

50 ml sterile glass tubes containing 10 ml of RPMI 1640 medium without fetal bovine serum).

Temperature:

Normal ambient temperature.

Sample Rejection Criteria:
Smelly or infected samples will be discarded.
Samples not put into the sterile tube during the process of biopsy will be discarded.
Sample not resected with fresh sterile blade will be discarded.
Patient suffering from any viral infections during the time of surgery (even in early or latent viraemia phase) will not be included. This will be checked by routine blood tests and serology panels before the patient is cleared for surgery.
Highly lipemic fluids will be discarded.

Transport:

Once the biopsy procedure is planned, the participating hospital will inform PMI's laboratory coordinator for efficient sample pick up. For emergency procedures after 7 μm, samples can be stored in room temperature for next day pick up.

### Opening:

The Samples received in the laboratory are opened in a vertical laminar air flow only.

### Example 2. Laboratory Method (Liver Organoid Development): DAY1

1. Take 10 coverslips.
2. Prepare 25-50 μg/ml collagen solution in ddH2O.
3. Add 1 μl of collagen solution in concentric circles in 15 spots.
4. Incubate for 1 hour at 37° C.
5. Rinse slide 3 times with ddH2O.
6. Dry the coverslip thoroughly.
7. Thaw a vial of normal hepatocyte cultured in the lab.
8. Add cells at a concentration of 70000 hepatocyte in a final volume of 50 μl of DMEM incomplete medium.
9. Put the culture in the incubator.
10. Every hour, take out the slide and shake it horizontally and vertically 3-4 times. 11. Check under microscope. 12. Wash unattached cells by gentle aspiration and with DMEM (incomplete). 13. Keep in 37° C. 5% CO2 overnight.

### DAY2:

1. Thaw fibroblast cells and HUVEC cells grown in the laboratory.
2. Incubate the culture with Fibronectin 2 mg/sqcm for 37° c.
3. Mix 6000 HUVEC with 90000 fibroblasts and seed this mixture onto hepatocytes at a ratio of 1:5.
4. Incubate for 4 hours.
5. Add a layer of matrigel to the cell mix.

### DAY3:

1. Add 6000 HUVEC cells on top of the Matrigel in DMEM (complete)
2. Keep to develop organoid for 3 days
   DAY7: 1. Add chemotherapy drugs to the organoids.
   DAY8: Collect the chemodrug containing medium containing active compounds and store in −80 C.

### I] Serum and ECM Extraction:

### A. Serum Extraction of the Patient:

Blood will be drawn from the patient by standard venepuncture method in a vacutainer, transferred to a clotted vial and allowed to clot in an upright position for 30 minutes (and not more than 60 minutes). Centrifugation will be performed for 15 minutes at 2500 rpm within one hour of collection, and the supernatent serum will be aliquoted and stored at −20° C.

### B. ECM Preparation:

1. The tumour pieces will be chopped with a surgical scalpel to 1 mm³ explants.
2. Tissue slices will be suspended in dispase solution, and incubated for 15 mins at 48 C.
3. The tissues will be homogenized in a high salt buffer solution containing 0.05M Tris pH 7.4, 3.4M sodium chloride, 4 mM of EDTA, 2 mM of N-ethylmaleimide and protease and phosphatase inhibitors using tissue homogenizer.
4. The homogenized mixture will be centrifuged three times at 7,000 g for 15 min and the supernatant will be discarded to retain the pellet. The pellet will be incubated in 2M Urea buffer (0.15M sodium chloride and 0.05M Tris pH 7.4) and stirred for 1 h at 50 C.
5. The complex extracted proteins will be solubilised in Urea buffer.
6. The mixture was then finally centrifuged at 14,000 g for 20 mins and re-suspended in the 2M Urea buffer, aliquoted and stored at −80 C. This protein solution is used as extracellular matrix protein for every individual patient.

### II] Primary Tumor 3D Culture:

DAY1:
1. Serum separation will be done from the blood collected from the patient.
2. Solid tumour will be minced to 96 small pieces using scalpel blades (S2646-100EA, Sigma)
3. From step1, tumour pieces will be put on to the wells according to the following layout:

| | | | | | | | | | | | |
|---|---|---|---|---|---|---|---|---|---|---|---|
| CON- | CON- | CON- | DRUG1 | DRUG1 | DRUG1 | DRUG1 | DRUG1 | DRUG1 | DRUG1 | DRUG1 | DRUG1 |
| TROL | TROL | TROL | DOSE1 | DOSE1 | DOSE1 | DOSE2 | DOSE2 | DOSE2 | DOSE3 | DOSE3 | DOSE3 |
| DRUG2 | DRUG2 | DRUG2 | DRUG2 | DRUG2 | DRUG2 | DRUG2 | DRUG2 | DRUG2 | DRUG3 | DRUG3 | DRUG3 |
| DOSE1 | DOSE1 | DOSE1 | DOSE2 | DOSE2 | DOSE2 | DOSE3 | DOSE3 | DOSE3 | DOSE1 | DOSE1 | DOSE1 |
| DRUG3 | DRUG3 | DRUG3 | DRUG3 | DRUG3 | DRUG3 | DRUG4 | DRUG4 | DRUG4 | DRUG4 | DRUG4 | DRUG4 |
| DOSE2 | DOSE2 | DOSE2 | DOSE3 | DOSE3 | DOSE3 | DOSE1 | DOSE1 | DOSE1 | DOSE2 | DOSE2 | DOSE2 |
| DRUG4 | DRUG4 | DRUG4 | DRUG5 | DRUG5 | DRUG5 | DRUG5 | DRUG5 | DRUG5 | DRUG5 | DRUG5 | DRUG5 |
| DOSE3 | DOSE3 | DOSE3 | DOSE1 | DOSE1 | DOSE1 | DOSE2 | DOSE2 | DOSE2 | DOSE3 | DOSE3 | DOSE3 |
| DRUG6 | DRUG6 | DRUG6 | DRUG6 | DRUG6 | DRUG6 | DRUG6 | DRUG6 | DRUG6 | DRUG7 | DRUG7 | DRUG7 |
| DOSE1 | DOSE1 | DOSE1 | DOSE2 | DOSE2 | DOSE2 | DOSE3 | DOSE3 | DOSE3 | DOSE1 | DOSE1 | DOSE1 |
| DRUG7 | DRUG7 | DRUG7 | DRUG7 | DRUG7 | DRUG7 | DRUG5 | DRUG5 | DRUG5 | DRUG5 | DRUG5 | DRUG5 |
| DOSE2 | DOSE2 | DOSE2 | DOSE3 | DOSE3 | DOSE3 | DOSE1 | DOSE1 | DOSE1 | DOSE2 | DOSE2 | DOSE2 |
| DRUG5 | DRUG5 | DRUG5 | DRUG9 | DRUG9 | DRUG9 | DRUG9 | DRUG9 | DRUG9 | DRUG9 | DRUG9 | DRUG9 |
| DOSE3 | DOSE3 | DOSE3 | DOSE1 | DOSE1 | DOSE1 | DOSE2 | DOSE2 | DOSE2 | DOSE3 | DOSE3 | DOSE3 |
| DRUG10 | DRUG10 | DRUG10 | DRUG10 | DRUG10 | DRUG10 | DRUG10 | DRUG10 | DRUG10 | CONTROL | CONTROL | CONTROL |
| DOSE1 | DOSE1 | DOSE1 | DOSE2 | DOSE2 | DOSE2 | DOSE3 | DOSE3 | DOSE3 | | | |

4. 10 μl of ECM material will be layered onto the wells surrounding the tumour piece.
5. 100 μl Complete RPMI1640 (with 10% patient's serum) will be added to each well.
6. Plates will be kept in 37° C. overnight.

DAY2: Chemotherapeutic drugs will be added (after organoid treatment)

DAY3: 30 μl of Complete RPMI1640 will be added to each well.

DAY4: Chemotherapeutic drugs will be added (after organoid treatment)

DAY5: 30 μl of Complete RPMI1640 will be added to each well.

DAY6: Chemotherapeutic drugs will be added (after organoid treatment)

DAY7: 30 μl of Complete RPMI1640 will be added to each well.

DAY8: (Parallel organoids will be started to grow in the lab like the liver organoid along with the first line experiment).

STEP1:

Medium will removed from the wells treated with same drugs and mixed together.

10 μl medium will be taken to count cells. Cells will be counted using trypan blue dye exclusion method. Total medium will be divided into 4 equal parts, and administered to 4 different 3D cultured organoids.

STEP2:

10% formalin (200 μl) will be added to 96 well plates, and the tissues will be fixed for 4 hours.

Formalin will be discarded, and FFPE will be prepared according to standard techniques.
3 μm sections will be cut on PL slides, and stained for H&E, Ki-67, and any other special stain if needed.

### III] Generating First Line Chemo Response Report:

All the sections will be evaluated for Chemo-induced necrosis and will be scored according to percent of cell necrosis.
Report will include:

1. Cell detachment function

2. Apoptosis percentage

3. Necrosis percentage

4. Best chemo option

5. 1st line chemotherapy resistance

### IV] Follow Up Patients Every 3 Months for Tumor Size, Morbidity Etc:

1. CT scan (with contrast) every 3 months

### 2. CBC, LFT, RFT

3. Charlson Co-morbidity index (CCI)

### V] Collection of Non-Adhering Chemo-Resistant Surviving Cells:

Using a pipette, 200 microlitre of medium containing non-adherent cells will be pooled from the same drug-treated wells as same treatment group irrespective of the dosing of chemotherapy given. Wells will be washed with PBS twice and pooled in the same drug treated group. Cells will be counted in a Neubauer hemocytometer using trypan blue dye exclusion method.

### DAY8 Continued:

Non-adhering chemo-resistant surviving cells will be given to different organoids in culture. The cultures will be maintained till day 23.

### DAY23:

Chemotherapeutic drugs will be added (after liver organoid treatment) (1st).

### DAY25:

30 μl of Complete DMEM/RPMI1640 will be added to each well.

### DAY27:

Chemotherapeutic drugs will be added (after liver organoid treatment) (2nd)

### DAY29:

30 μl of Complete DMEM/RPMI1640 will be added to each well.

### DAY31:

Chemotherapeutic drugs will be added (after liver organoid treatment)(3rd)

### DAY33:

30 μl of Complete DMEM/RPMI1640 will be added to each well.

### DAY34:

10% formalin (200 μl) will be added, and the tissues will be fixed for 4 hours.
Formalin will be discarded, and FFPE will be prepared according to standard techniques.
3 μm sections will be cut on PL slides, and stained for H&E, Ki-67, and any other special stain if needed.
Report will include:

Apoptosis percentage

Necrosis percentage

Best chemo option

### VI] Generation of Second Line Chemo Report.

10% formalin (200 μl) will be added, and the tissues will be fixed for 4 hours.
Formalin will be discarded, and FFPE will be prepared according to standard techniques.
3 μm sections will be cut on PL slides, and stained for H&E, Ki-67, and any other special stain if needed.
Report will include:

Apoptosis percentage

Necrosis percentage

Best 2^{nd} line chemo option

### VII] Follow Up of Patients at Every 3 Month Interval for 2 Year:

Follow up will be done with CT scans (contrast enhanced), CBC, LFT, RFT, and Charlson Co-morbidity index (CCI) every 3 months for 2 years after the initiation of chemotherapy.
VIII] Correlation Curves with Laboratory Generated Data and Patient Data Will be Accrued to Generate Statistical Data for Predicting Time to Recurrence and Organ of Metastasis.

The entire screening procedure may require 2-3 weeks to complete, depending upon the cell growth demonstrated after the first-line chemotherapy. However, at the end of that period, the clinician has already identified the most appropriate second-line chemotherapeutic agent to use for a particular patient, should the cancer recur in that patient.

In one embodiment of the invention, the presently disclosed screening procedure may include a method of screening chemotherapeutics for second-line chemotherapy, where the method comprises:

culturing cancer cells of interest to prepare plural first cultures;

exposing each of the first cultures to one of a plurality of first chemotherapeutic agents;

identifying a most effective member of the plurality of first chemotherapeutic agents;

culturing cancer cells that survived exposure to the most effective first chemotherapeutic agent to prepare plural second cultures;

exposing each of the second cultures of cancer cells to one of a plurality of second chemotherapeutic agents; and

identifying a most effective member of the plurality of second chemotherapeutic agents.

Each of the first and second cultures of the method may be prepared on a substrate.

The substrate may include a biological polymer.

The substrate may include a proteinaceous matrix.

The substrate may include a monolayer of normal cells.

The normal cells and cancer cells of interest may be collected from a single patient.

The plurality of first and/or second chemotherapeutic agents may include one or more of paclitaxel, carboplatin, cisplatin, adriamycin, gemcitabine, topotecan, etoposide, docataxel, ifosamide, and 5-fluoro uracil.

The method of screening may be performed using a multiwall microplate.

In another embodiment of the invention, the presently disclosed screening procedure may include a method comprising:

preparing plural substrates, each substrate including a layer of normal cells on an organic matrix;

culturing cancer cells of interest on the prepared substrates to prepare plural first cultures; and

exposing each of the first cultures to one of a plurality of first chemotherapeutic agents.

The presently disclosed assay provides significant advantages over currently available chemopredictive assays. In particular, where an appropriate substrate is used, the disclosed chemopredictive assay provides an authentic ex vivo environment, such as where the substrate includes an extracellular matrix and/or the use of normal cells obtained from the patient of interest in the region where the tumor exists.

Although the present invention has been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances.

What is claimed:

1. A method of screening chemotherapeutics for second-line chemotherapy, comprising:
culturing cancer cells of interest to prepare plural first cultures;
exposing each of the first cultures to one of a plurality of first chemotherapeutic agents;
identifying a most effective member of the plurality of first chemotherapeutic agents;
culturing cancer cells that survived exposure to the most effective first chemotherapeutic agent to prepare plural second cultures;
exposing each of the second cultures of cancer cells to one of a plurality of second chemotherapeutic agents; and
identifying a most effective member of the plurality of second chemotherapeutic agents.

2. A method of screening chemotherapeutics for second-line chemotherapy, comprising:
preparing plural substrates, each substrate including a layer of normal cells on an organic matrix;
culturing cancer cells of interest on the prepared substrates to prepare plural first cultures; and
exposing each of the first cultures to one of a plurality of first chemotherapeutic agents.
